(19) United States
(12) Patent Application Publication
(10) Pub. No.: US 2023/0272331 A1
(43) Pub. Date: Aug. 31, 2023

(54) Predictive Modeling and Control of Cell Culture

(71) Applicant: AMGEN INC., Thousand Oaks, CA (US)

(72) Inventors: Hamid Khodabandehlou, Thousand Oaks, CA (US); Tony Y. Wang, Tiverton, RI (US); Aditya Tulsyan, Atlanta, GA (US)

(21) Appl. No.: 18/029,715

(22) PCT Filed: Sep. 22, 2021
(86) PCT No.: PCT/US21/51570

§ 371 Date: Mar. 31, 2023

Related U.S. Application Data
(60) Provisional application No. 63/086,417, filed on Oct. 1, 2020.

(51) Int. Cl. C12M 1/36; G06N 3/02; C12M 1/34

(52) U.S. Cl. CPC C12M 41/48; G06N 3/02; C12M 41/32

(57) ABSTRACT

A method of controlling a cell culture process includes, for each time interval of one or more time intervals during the cell culture process, obtaining current values of one or more cell culture attributes associated with a cell culture, predicting one or more future values of a particular cell culture attribute associated with the cell culture, and controlling one or more physical inputs to the cell culture process. Predicting the future value(s) includes applying the current values of the cell culture attribute(s), and an earlier value of at least one of the cell culture attributes, as inputs to a data-driven predictive model using historical data. Controlling the physical input(s) includes applying the future value(s) as inputs to a model predictive controller.

### FIELD OF THE DISCLOSURE

The present application relates generally to cell cultures (e.g., in a bioreactor), and more specifically to the prediction and control of cell culture attributes (measured or non-measured) based on measured cell culture attribute values.

### BACKGROUND

In the manufacture of certain biopharmaceutical products (e.g., biotherapeutic proteins), bioreactors are used to culture cells prior to harvesting a desired drug product. Stable production of such drug products generally requires that a bioreactor maintain balanced and consistent parameters (e.g., cellular metabolic concentrations), which in turn demands rigorous process monitoring and control. Because the cell culture environment is dynamic and complex, however, it is generally difficult to apply physical inputs to the cell culture process (e.g., feed volumes, temperatures, glucose infusions, etc.) in a manner that will result in the desired cell culture attributes (e.g., viable cell density, glucose level, etc.). Various efforts to optimize the cell culture process have been made, including efforts to model, and control the physical inputs to, the cell culture process. However, while some progress has been made, modeling and control remain a significant challenge due to the complex, nonlinear behaviors of the cell culture process, the lack of relevant measurements, and the lack of available experimental data.

Conventionally, nutrient levels in bioreactors are controlled either manually or using traditional proportional-integral-derivative (PID) controllers via bolus feeds (see Mehdizadeh et al., Generic Raman-Based Calibration Models Enabling Real-Time Monitoring of Cell Culture Bioreactors, Biotechnol. Prog. 31(4), pp. 1004-1013 (2015)). Although manual and PID controllers have produced acceptable results, the manufacturing process still contains many opportunities for further optimization, e.g., to maximize growth, yield or optimally control product quality. Mechanistic, mathematical models have also been proposed to model bioprocesses. For example, a first-principles, mechanistic model has been proposed along with a model-predictive controller (MPC) to control the glucose level in a pilot plant bioreactor (see Craven et. al., Glucose Concentration Control of a Fed-Batch Mammalian Cell Bioprocess Using a Nonlinear Model Predictive Controller, Journal of Process Control, 24(4), pp. 359-366 (2014)), where the model used the rate of change of process state variables and a nonlinear MPC to control the bioreactor. In that approach, multiple, first-order ordinary differential equations are used to model the bioreactor. However, to obtain an accurate final model of the bioreactor, this mechanistic approach requires applying extensive knowledge of the process to the model, which results in a complex, hybrid model.

### BRIEF SUMMARY

Systems and methods described herein generally use historical measurements and machine learning to build a dynamic model of a cell culture process. For example, historical (measured) metabolite concentrations from real-world cell culture processes may be used to train a dynamic, data-driven predictive model. When applied to a real-world process, the model can predict future cell culture attributes (e.g., future metabolite levels) based on current (e.g., real-time) measurements of the cell culture. The model also makes use of measurements taken in one or more earlier time intervals (e.g., by using metabolite levels measured on the current day and also on one or more days prior to the current day). The model may be a neural network or a regression model, for example. The predictions output by the model may be input to a model-predictive controller (MPC), where the MPC has an objective function set to maximize a desired cell culture attribute (e.g., viable cell density, total cell density, etc.). The MPC can then take the appropriate control action or actions (e.g., add glucose to the bioreactor) to manage and control the cell culture in a manner that guides the cell culture process to the desired objective (e.g., maximum viable cell density, etc.).

The techniques disclosed herein may obviate the need for manual sampling of cell culture attributes and/or manual adjusting of control set-points. Moreover, by accounting for nonlinear cell culture behaviors and historical measurement values, these techniques may provide improved prediction accuracy relative to other modeling and control techniques. A dynamic process model with MPC may allow for bi-directional flow of data with the capability to adjust and learn the cell culture process in real-time.

### BRIEF DESCRIPTION OF THE DRAWINGS

The skilled artisan will understand that the figures described herein are included for purposes of illustration and are not limiting on the present disclosure. The drawings are not necessarily to scale, emphasis instead being placed upon illustrating the principles of the present disclosure. It is to be understood that, in some instances, various aspects of the described implementations may be shown exaggerated or enlarged to facilitate an understanding of the described implementations. In the drawings, like reference characters throughout the various drawings generally refer to functionally similar and/or structurally similar components.

FIG. 1 is a simplified block diagram of an example system that may be used to monitor and control a cell culture process.

FIG. 2 is a block diagram of an example model that may be implemented in the system of FIG. 1.

FIG. 3 depicts example operation of a model-predictive controller that may be used as the model-predictive controller of FIG. 1 and/or FIG. 2.

FIG. 4 depicts an example sequence of predictions made by the predictive model of FIG. 1 and/or FIG. 2.

FIG. 5 depicts an example neural network that may be used as the predictive model of FIG. 1 and/or FIG. 2.

FIGS. 6A-6E are example plots comparing measured and predicted values of different cell culture attributes when using a neural network and a truncated set of metabolite measurements.

FIGS. 7A-7E are example plots comparing measured and predicted values of different cell culture attributes when using a second-order regression model and a truncated set of metabolite measurements.

FIGS. 8A-8E are example plots comparing measured and predicted values of different cell culture attributes when using a third-order regression model and a truncated set of metabolite measurements.

FIGS. 9A-9J are example plots comparing measured and predicted values of different cell culture attributes when using a neural network and a full set of metabolite measurements.

FIGS. 10A-10J are example plots comparing measured and predicted values of different cell culture attributes when using a second-order regression model and a full set of metabolite measurements.

FIGS. 11A-11J are example plots comparing measured and predicted values of different cell culture attributes when using a third-order regression model and a full set of metabolite measurements.

FIG. 12 is a flow diagram of an example method of controlling a cell culture process.

### DETAILED DESCRIPTION

The various concepts introduced above and discussed in greater detail below may be implemented in any of numerous ways, and the described concepts are not limited to any particular manner of implementation. Examples of implementations are provided for illustrative purposes.

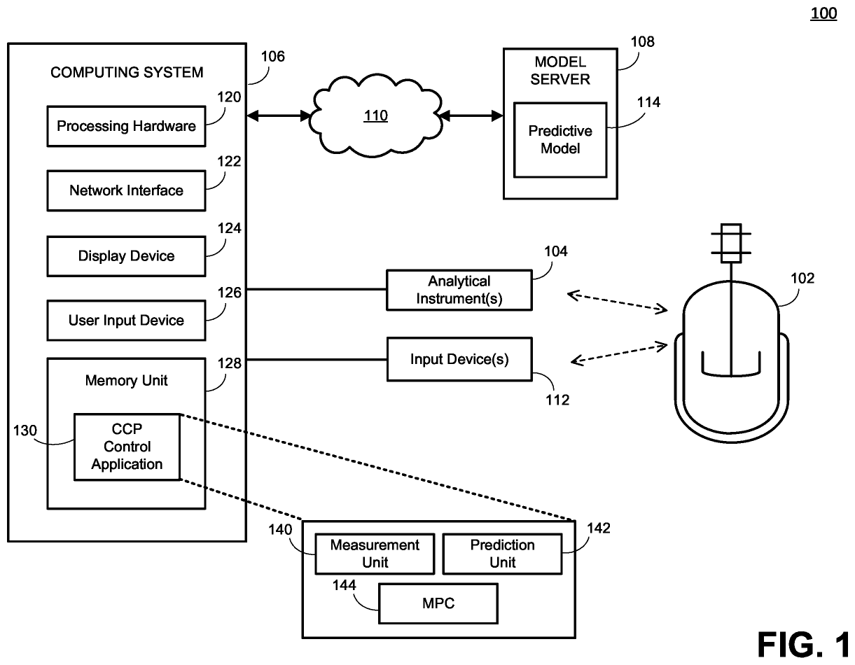

FIG. 1 is a simplified block diagram of an example system 100 that may be used to manually monitor and control a cell culture process. The system 100 includes a bioreactor 102, one or more analytical instruments 104, a computing system 106, a model server 108, a network 110, and one or more input devices 112.

The bioreactor 102 may be any suitable vessel, device or system that supports a cell culture, which may include living organisms and/or substances derived therefrom within a media. The bioreactor 102 may contain recombinant proteins that are being expressed by the cell culture, e.g., such as for research purposes, clinical use, commercial sale, or other distribution. Depending on the biopharmaceutical process being monitored, the media may include a particular fluid (e.g., a “broth”) and specific nutrients, and may have a target pH level or range, a target temperature or temperature range, and so on.

The analytical instrument(s) 104 are communicatively coupled to the computing system 106, and may include any in-line, at-line and/or off-line instrument, or instruments, configured to measure one or more attributes of the cell culture within the bioreactor 102. For example, the analytical instrument(s) 104 may measure one or more media component concentrations, such as metabolite levels (e.g., glucose, lactate, sodium, potassium, glutamine, ammonium, etc.). Additionally or alternatively, the analytical instrument(s) 104 may measure osmolality, viable cell density (VCD), total cell density (TCD), viability, and/or one or more other cell culture attributes associated with the contents of the bioreactor 102.

While in some embodiments the analytical instrument(s) 104 may use destructive analysis techniques, in other embodiments one, some, or all of the analytical instrument(s) 104 use non-destructive analysis (e.g., “soft sensing”) techniques. For example, the analytical instrument(s) 104 may include a Raman analyzer with a spectrograph and one or more probes. The Raman analyzer may include a laser light source that delivers the laser light to the probe(s) via respective fiber optic cables, and may also include a charge-coupled device (CCD) or other suitable camera/recording device to record signals that are received from probe(s) via other channels of the respective fiber optic cables. Alternatively, the laser light source(s) may be integrated within the probe(s). Each probe may be an immersion probe or any other suitable type of probe (e.g., a reflectance probe or transmission probe). The analyzer and probe(s) may non-destructively scan for the relevant cell culture attribute within the bioreactor 102 by exciting, observing, and recording a molecular “fingerprint” of the cell culture process. The molecular fingerprint corresponds to the vibrational, rotational and/or other low-frequency modes of molecules within the biologically active contents when those contents are excited by the laser light delivered by the probe(s). As a result of this scanning process, the Raman analyzer generates one or more Raman scan vectors that each represent intensity as a function of Raman shift (frequency). The Raman analyzer may then analyze the Raman scan vector(s) in order to determine (e.g., infer) values of corresponding cell culture attributes (e.g., glucose and/or other metabolite concentrations).

The input device(s) 112 are also communicatively coupled to the computing system 106, and may be any device or devices that delivers a physical input to the contents of the bioreactor 102. For example, the input device(s) 112 may include a glucose pump, a device that adds a controlled amount of feed to the bioreactor, and/or a device that provides heat and/or cooling to the bioreactor 102 and its contents. Generally, the input device(s) 112 may include pumps, valves, and/or any other suitable type(s) of control element(s). The input device(s) 112 may include proportional-integral-derivative (PID) controllers, and receive set-points from the computing system 106 as inputs to the PID controllers, for example.

The model server 108 includes processing hardware and memory (not shown in FIG. 1), and stores a predictive model 114 that will be discussed in further detail below. The functionality of the model server 108 described herein may be provided by the processing hardware of the model server 108 when executing instructions stored in the memory of the model server 108, for example. The network 110 couples the model server 108 to the computing system 106, and may be a single communication network or may include multiple communication networks of one or more types (e.g., one or more wired and/or wireless local area networks (LANs), and/or one or more wired and/or wireless wide area networks (WANs) such as the Internet or an intranet, for example).

The computing system 106 may be a server, a desktop computer, a laptop computer, a tablet device, or any other suitable type of computing device or devices. In the example embodiment shown in FIG. 1, the computing system 106 includes processing hardware 120, a network interface 122, a display device 124, a user input device 126, and a memory unit 128. In some embodiments, however, the computing system 106 includes two or more computers that are either co-located or remote from each other. In these distributed embodiments, the operations described herein relating to the processing hardware 120, the network interface 122, and/or the memory unit 128 may be divided among multiple processing units, network interfaces, and/or memory units, respectively.

The processing hardware 120 includes one or more processors, each of which may be a programmable microprocessor that executes software instructions stored in the memory unit 128 to execute some or all of the functions of the computing system 106 as described herein. Alternatively, some of the processors in the processing hardware 120 may be other types of processors (e.g., application-specific integrated circuits (ASICs), field-programmable gate arrays (FPGAs), etc.), and some of the functionality of the computing system 106 as described herein may instead be implemented, in part or in whole, by such hardware. The memory unit 128 may include one or more physical memory devices or units containing volatile and/or non-volatile memory. Any suitable memory type or types may be used, such as read-only memory (ROM), solid-state drives (SSDs), hard disk drives (HDDs), and so on.

The network interface 122 may include any suitable hardware (e.g., front-end transmitter and receiver hardware), firmware, and/or software configured to communicate via the network 110 using one or more communication protocols. For example, the network interface 122 may be or include an Ethernet interface.

The display device 124 may use any suitable display technology (e.g., LED, OLED, LCD, etc.) to present information to a user, and the user input device 126 may be a keyboard or other suitable input device. In some embodiments, the display device 124 and the user input device 126 are integrated within a single device (e.g., a touchscreen display). Generally, the display device 124 and the user input device 126 may jointly enable a user to interact with graphical user interfaces (GUIs) provided by the computing system 106, e.g., for purposes such as monitoring the cell culture process occurring within the bioreactor 102. In some embodiments, however, the computing system 106 does not include the display device 124 and/or the user input device 126.

The memory unit 128 stores the instructions of one or more software applications, including a cell culture process (CCP) control application 130. The CCP control application 130, when executed by the processing hardware 120, is generally configured to communicate with the analytical instrument(s) 104, the model server 108, and the input device(s) 112 to obtain measured (or measurement-based) values of cell culture attributes, predict future values of cell culture attributes based on the measured/obtained values, and control one or more inputs to the cell culture process based on the predicted future values (e.g., all in real-time). To this end, the CCP control application 130 includes a measurement unit 140, a prediction unit 142, and a model-predictive controller (MPC) 144. It is understood that the various units of the CCP control application 130 may be distributed among different software applications, and/or that the functionality of any one such unit may be divided among different software applications.

The measurement unit 140 may obtain (e.g., request, or otherwise monitor) the measurements produced by the analytical instrument(s) 104 once per time interval for any desired number of time intervals (e.g., once per day, once per hour, etc.). In some embodiments, the measurement unit 140 determines one or more cell culture attribute values by processing values obtained from the analytical instrument(s) 104. For example, the measurement unit 140 may determine an average metabolite concentration once per time interval (e.g., once per day) based on metabolite measurements provided on a more frequent basis (e.g., once every 15 minutes, once per hour, etc.) by one of the analytical instrument(s) 104. As another example, the measurement unit 140 may analyze Raman scan vectors provided by a spectrograph of analytical instrument(s) 104 to determine or infer the values of one or more cell culture attributes (as discussed above). In some embodiments, the measurement unit 140 (or another unit, application, device, or computing system) determines cell culture attribute values (e.g., metabolite concentrations) based on Raman scan vectors using just-in-time learning (JITL), according to any of the embodiments described in PCT Patent Application No. PCT/US2019/057513, filed on Oct. 23, 2019 and entitled “Automatic Calibration and Automatic Maintenance of Raman Spectroscopic Models for Real-Time Predictions,” the disclosure of which is hereby incorporated by reference herein in its entirety. Generally, as used herein for ease of explanation, terms such as “measured” and “measurement” are broadly used to refer to a physically/directly measured value, a soft-sensed value, or a value derived from (e.g., calculated using) a physically/directly measured or soft-sensed value, unless the context of their use clearly indicates a more specific meaning.

The prediction unit 142 may provide measured values of cell culture attributes to the model server 108, by causing the computing system 106 to transmit the measurement data to the model server 108 via the network interface 122 and the network 110. The model server 108 then applies the cell culture attribute values as inputs to the predictive model 114. The predictive model 114 is a data-driven, machine learning model that predicts one or more future values of at least one cell culture attribute based on the model inputs. The model inputs include one or more current measured values of cell culture attributes for each time interval, as well as measured values from one or more previous time intervals or a simulated value for at least one of those cell culture attributes. For example, the predictive model 114 may predict a future glucose concentration (e.g., at each of x future time intervals, where x is an integer greater than zero) based on glucose concentrations measured on the current day and the previous day. As another example, the predictive model 114 may predict a future VCD, TCD, or viability (e.g., at each of x future time intervals, where x is an integer greater than zero) based on various metabolite concentrations measured on the current day and each of the past two days.

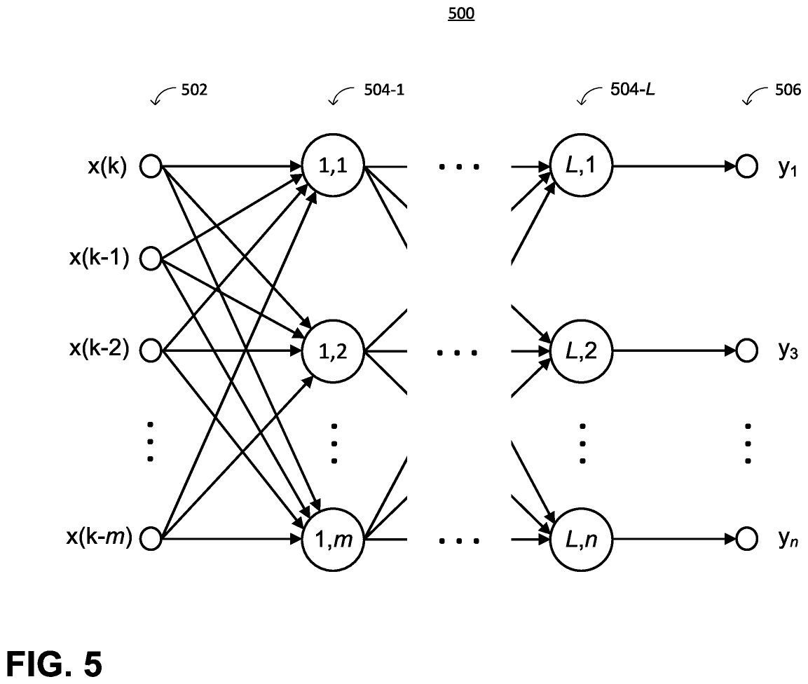

The predictive model 114 may be a neural network, such as a feedforward neural network or recurrent neural network, that was trained using historical values of the measured cell culture attributes (i.e., model inputs) and the corresponding real-world results (i.e., labels for supervised training). Examples of such neural networks are is discussed below in connection with FIG. 5.

In other embodiments, the predictive model 114 is a regression model. For example, the predictive model 114 may be a second-order, third-order, or higher-order (fourth, fifth, sixth, etc.) regression model or a combination of different order regression models. As used herein, the term “order” refers to the maximum number of different time intervals reflected in the measurements that are used as the model inputs when forming a prediction of one or more future time interval values. For the sake of clarity, a regression model that uses a n-th order regression model at least once (in a model employing orders including n and one or more of n−1, n−2 . . . ) would be considered an n-th order regression model. In one example, a model using both second-order models and third-order models would be considered a third-order regression model. Thus, for example, a regression model that operates on current day and previous day measured metabolite concentrations would be referred to as a second-order regression model, while a regression model that operates on metabolite concentrations from the current day, the previous day, and the day before the previous day would be referred to as a third-order regression model. More generally, a second-order regression model would, for at least one cell culture attribute used as a model input, operate on measurements obtained at time intervals k and (k-x), and a third-order regression model would, for at least one cell culture attribute used as a model input, operate on measurements obtained at time intervals k, (k-x), and (k-y), where x is any integer greater than zero and y is any integer greater than x. The regression model 114 may be linear or non-linear, depending on the embodiment.

Because third-order regression models require measurements from two earlier time intervals (e.g., the two preceding days), it may not be possible to use such a model for the first two time intervals (e.g., Day 0 and Day 1). In some embodiments, therefore, the prediction unit 142 of FIG. 1 uses a second-order regression model initially (e.g., starting at the second time interval), and then switches to a third-order regression model thereafter (e.g., starting at the third time interval). In other embodiments, the prediction unit 142 uses other techniques for the initial time interval(s) (e.g., at the first time interval, simply setting two “previous measurement” values equal to the current measurement value).

Other model types are also possible. However, neural networks and higher-order regression models can provide advantages over certain other model types. For example, support vector regression models, Gaussian process regression models, and random forest regression models have been found to have a relatively large prediction error. Further, echo state networks (ESNs) have been found to be highly sensitive to measurement error, although such models may be suitable for prediction at the initial time interval (e.g., day one).

The model server 108 may execute the predictive model 114 and exchange data with the computing system 106 as part of a web services model, for example. In other embodiments, however, the system 100 does not include the model server 108, and the computing system 106 locally stores (and possibly trains) the predictive model 114 (e.g., in the memory unit 128), and locally executes the predictive model 114 (e.g., by the processing hardware 120 when executing the instructions of the prediction unit 142). In the example embodiment shown, for each time interval, the model server 108 applies the relevant inputs (measured cell culture attribute values) to the predictive model 114, and returns the predicted future value(s) to the prediction unit 142 via the network 110. The CCP control application 130 may store the future value(s) within the memory unit 128 (or another suitable memory), and applies the future value(s) (or values that the CCP control application 130 derives therefrom) as inputs to the MPC 144.

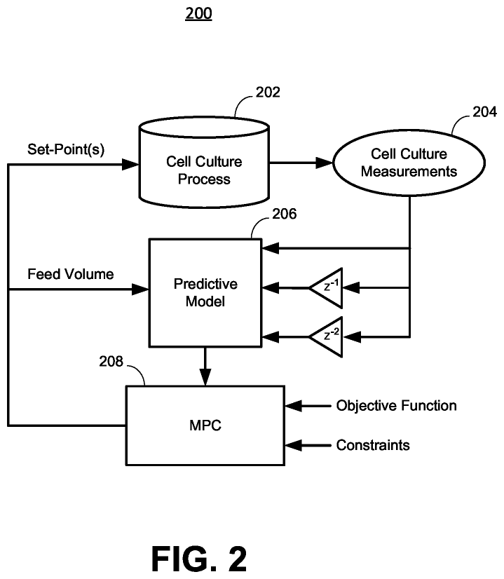

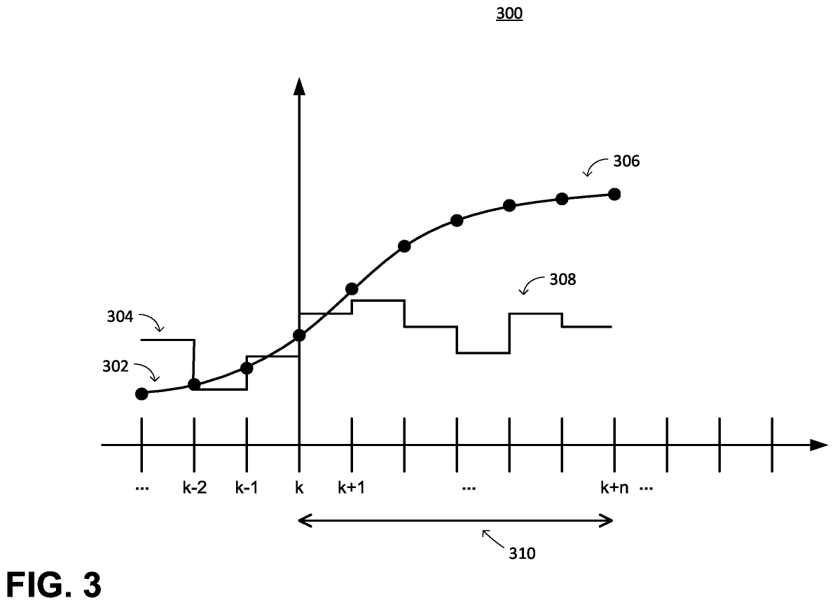

The MPC 144 operates on the predicted future cell culture attribute value(s), and possibly also other information, to generate a control signal for one of the input device(s) 112. For example, the computing system 106 may send, to a glucose pump of the input device(s) 112, a command that conforms to a protocol recognized by the pump and specifies a desired glucose infusion amount (or infusion time period, etc.) that the pump is to add or apply to the contents of the bioreactor 102. MPCs that may be used as the MPC 144 are discussed in further detail below with reference to FIGS. 2 and 3.

The model server 108 may store only one predictive model 114, or may store multiple predictive models that each output/predict future values for a different cell culture attribute. In the latter case, the various models may operate on the same or different sets of model inputs, depending on the embodiment. The predictive models may be of the same type (e.g., all third-order regression models), or different types (e.g., a feedforward neural network for predicting VCD, and third-order regression models for predicting glucose and lactate concentrations). In each of these embodiments, the prediction unit 142 may provide the necessary inputs (e.g., current and past cell culture attribute values) to the model server 108 via the network 110, and the model server 108 may execute the predictive models to predict (and return to computing system 106) one or more future values for each of the different cell culture attributes being predicted (e.g., VCD, TCD, glucose concentration, etc.). The MPC 144 in such embodiments may include one MPC per predicted cell culture attribute (e.g., one MPC for VCD, one for TCD, etc.), with each MPC generating a control signal for a different one of the input devices 112. Alternatively, the CCP control application 130 apply predicted values for multiple cell culture attributes to a single MPC. While the description that follows focuses on the predicted future values of a single cell culture attribute being used as inputs to the single MPC 144, it is understood that there may be additional predicted cell culture attributes and/or additional MPCs.

In some embodiments, the CCP control application 130 also arranges for the presentation (to a user) of information such as the measured values (e.g., the inputs to the predictive model 114) and/or the future values output by the predictive model 114 (e.g., to enable concurrent manual monitoring/oversight of the cell culture process). For example, the CCP control application 130 may generate and/or populate a graph showing past, current, and predicted/future values of cell culture attributes, and cause the display device 124 to display the graph. Alternatively or additionally, the CCP control application 130 may cause the display device 124 to show the values in a table format, and/or in some other suitable format. In still other embodiments, the CCP control application 130 is not responsible for displaying any information to any user.

It is understood that other configurations and/or components may be used instead of those shown in FIG. 1. For example, a different computing device or system (not shown in FIG. 1) may transmit measurements provided by the analytical instrument(s) 104 to the model server 108, one or more additional computing devices or systems may act as intermediaries between the computing system 106 and the model server 108, some of the functionality of the computing system 106 as described herein may instead be performed remotely by the model server 108 and/or another remote server, and so on.

FIG. 2 is a block diagram of an example architecture 200 that may be implemented in a system such as the system 100 of FIG. 1. In FIG. 2, a cell culture process 202 takes place in a vessel (e.g., in the bioreactor 102 of FIG. 1). Various cell culture measurements 204 (i.e., measured cell culture attribute values) are obtained using one or more instruments, such as the analytical instrument(s) 104 of FIG. 1. The cell culture measurements 204 may include concentrations of one, some, or all of a set of metabolites in the cell culture (e.g., glucose, lactate, sodium, potassium, ammonium, and/or glutamine). In some embodiments, the cell culture measurements 204 also (or instead) include one or more other types of measured cell culture attributes, such as VCD, TCD, viability, osmolality, etc.

The cell culture measurements 204 are provided as inputs (e.g., by the prediction unit 142 and/or by the model server 108) to a predictive model 206, which may be the same as the predictive model 114 of FIG. 1, for example. In FIG. 2, delay elements (z−1 and z−2) are used to indicate that past cell culture measurements 204 are also provided as inputs to the predictive model 206. FIG. 2 shows a third-order (regression or neural network) model embodiment in which the predictive model 206 operates on values from the current time interval (e.g., current day, or current hour, etc.) and the previous two time intervals (e.g., past two days) for all of the cell culture measurements 204. In other embodiments, however, past values are only used for a subset of the cell culture measurements 204, and/or the predictive model is of a different order (e.g., second-order, fourth-order, etc.).

At each time interval, the predictive model 206 processes the model inputs (i.e., current and past measured values) to generate predicted values of a cell culture attribute over a finite control horizon (e.g., the next four time intervals, or the next six time intervals, etc.) of the MPC 208. The attribute for which the future value(s) are predicted may be an attribute that was also measured and used as a model input, or may be an attribute that was not measured and used as a model input, depending on the embodiment. The predictive model 206 may predict future metabolite (e.g., glucose) concentrations, for example. In some embodiments, the architecture 200 includes multiple predictive models that are similar to the predictive model 206, but each predict values of a different cell culture attribute.

At each time interval, the example MPC 208 operates on the predicted future value(s), and possibly also other information, to generate a control signal (e.g., a set-point) for at least one of the input device(s) 112, e.g., using predictive batch-trajectory optimization. The MPC 208 may apply the measured/input cell culture attribute value(s) as independent variables of an objective function with one or more terms, with the dependent variable(s) of the objective function being the predicted value(s). The MPC 208 may then determine the optimal independent variable value(s), e.g., the value(s) that minimize the objective (or “cost”) function, subject to a number of constraints on the dependent and/or independent variables. Constraints may include, for example, “zero” as a minimum metabolite concentration, a maximum infusion rate associated with a glucose pump, and/or other suitable constraints. The objective function may include one term, or multiple terms (e.g., one term for each of multiple process inputs that the CCP control application 130 is controlling, such as glucose concentration, added feed volume, etc.), set such that optimization is achieved when the corresponding cell culture attribute (e.g., metabolite concentration) reaches some desired, pre-determined value, or to maximize a particular cell culture attribute (e.g., VCD, TCD, viability, etc.). The MPC 208 may then determine the control set-point to guide the cell culture process to the desired objective (e.g., maximized VCD, etc.).

In the example of FIG. 2, the MPC 208 provides one or more set-points including feed volume added to the bioreactor (e.g., bioreactor 102), with the MPC 208 providing the feed volume not only to the cell culture process 208 (or more precisely, to an input device that controls the amount of added feed), but also to the predictive model 206. The predictive model 206, in this example, uses the feed volume set-point as one of the model inputs along with the cell culture measurements 204.

FIG. 3 depicts the operation 300 of an example MPC in a particular embodiment and scenario. In FIG. 3, the x-axis represents time intervals (k, k+1, etc.) while the y-axis represents amplitudes (e.g., set-point values, etc.). The area to the left of the y-axis (k−1, k−2, etc.) represents past time intervals, the area to the right of the y-axis (k+1, k+2, etc.) represents future time intervals, and k represents the current time interval. FIG. 3 shows past (measured) cell culture attribute values 302 and past control set-points 304, as well as future cell culture attribute values 306 predicted by the predictive model (e.g., predictive model 142 or 206) and future control set-points 308 calculated by the MPC (e.g., MPC 144 or 208) over a finite control horizon 310.

In this particular example, the finite control horizon 310, and equivalently the prediction horizon of the predictive model (e.g., predictive model 206) covers n future time intervals, where n can be any suitable integer greater than one. In some embodiments, because the first few predictions should be more reliable, the MPC (e.g., MPC 144 and/or 208) has a prediction horizon of only four days, or only three days, etc. A shorter prediction horizon and shorter finite control horizon 310 may provide stability if the system experiences a disturbance, and/or may optimize the system to get the best final result. Because the system will continue to obtain new cell culture measurements, the predictions for the later days may improve over time. Model improvements may be made to achieve prediction horizons above four days (e.g., six days, etc.), particularly when the system is at steady-state such as in a continuous manufacturing (CM) process.

Returning now to FIG. 2, in some embodiments, the cell culture measurements 204 include values generated using just-in-time learning (JITL), as discussed above with reference to FIG. 1. In some of these embodiments, the JITL outputs may be input to the predictive model 206. In alternative embodiments, the JITL outputs may be input to a dynamic mode decomposition (DMD) or sparse identification of non-linear dynamics (SINDY) model. The model may be an ODE based model, for example. The DMD/SINDY model may in turn provide its outputs to the MPC 208, which may utilize a GEKKO optimizer, for example.

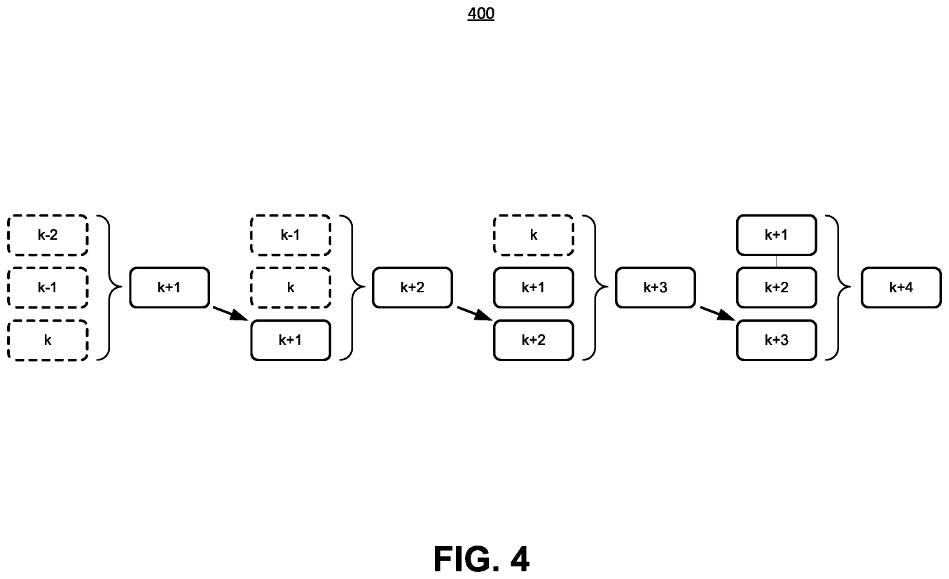

FIG. 4 depicts an example sequence 400 of predictions made by the predictive model 142 of FIG. 1 and/or the predictive model 206 of FIG. 2. In FIG. 4, the parameter k represents the current time interval. Thus, in an example where each time interval is one day, k is the current day, k+1 is the next day, k−1 is the previous day, and so on. The sequence 400 shows the prediction progress of a third-order predictive model (e.g., neural network or third-order regression model) for the current time interval k. Boxes with a dashed outline represent analytical measurements (e.g., time intervals at which the analytical instrument(s) 104 take the measurements), while boxes with solid outlines represent values predicted by the third-order predictive model. As seen in this example, analytical measurements of a cell culture attribute for the current (k) and past two (k−1 and k−2) time intervals are input to the predictive model (possibly along with measurements of other cell culture attributes), which allow the predictive model to predict the value of the cell culture attribute at the next time interval k+1. The predictive model then uses the predicted value for time interval k+1, along with the measured values fork and k−1, to predict the value of the cell culture attribute at the next time interval k+2. The predictive model then uses the predicted values for time intervals k+1 and k+2, along with the measured value for k, to predict the value of the cell culture attribute at the next time interval k+3, and so on, out to a prediction horizon of four time intervals (to k+4), in this example.

As noted above, the predictive model (e.g., the predictive model 142 of FIG. 1 and/or the predictive model 206 of FIG. 2) may be a neural network or a higher-order (second-order or higher) regression model. A simplified example of a neural network 500 is shown in FIG. 5. Neural networks are proven general function approximators. That is, a neural network can approximate any nonlinear input-output behavior by manipulating the number of layers and the availability of training data, and by using the appropriate training method. As seen in FIG. 5, the neural network 500 includes a number of inputs in an input layer 502, internal nodes at each of a number of internal or hidden layers 504-1 through 504-L (with L being any suitable integer greater than zero), and a number of outputs in an output layer 506. In this example, the neural network 500 is an (m+1)-th order neural network that operates on inputs from the current day or other time interval (x(k)) as well as each previous time interval back to (and including) the previous m-th time interval x(k−m), where m is any suitable integer greater than zero. While FIG. 5 shows n outputs in layer 506, in some embodiments the neural network 500 only includes a predicted value at the next time interval (i.e., y(k+1)). A prediction sequence similar to sequence 400 of FIG. 4 may then be used to run multiple iterations of the neural network 500, thereby generating additional predicted values (e.g., y(k+2), y(k+3), etc.) over the length of the desired prediction/finite control horizon.

The governing equation of the neural network 500 can be expressed as:

ŷ(k)=Uφ(W(x(k−1))) (Equation 1)

In Equation 1, x(k) and ŷ(k) are network input vectors applied at layer 502 and network output vectors produced at layer 506, respectively, and U and W are network weight matrices found by optimizing the training cost function. The neural network training cost function can be assumed to be a traditional “sum of squared errors” (SSE):

$$J = \frac{1}{2} {\sum }_{k = 1}^{N} {‖ y ( k ) - \hat{y} ( k ) ‖}^{2} \qquad ( Equation 2 )$$

In Equation 2, y(k) is the measured output and N is the number of training samples. Various local and global optimization approaches have been proposed to find network weight parameters by optimizing the training cost function such as the function of Equation 2. While local optimization approaches are relatively fast, they tend to be trapped in local minima of the optimization problem, which leads to poor generalization performance. In some embodiments, a scaled conjugate gradient approach is used to optimize the training cost function and find the network weight parameters. “Scaled conjugate gradient” is a fast and automated training algorithm that, unlike many other training algorithms, does not have any user-dependent parameters and is less likely to be trapped in the local minima of the optimization problem. In some embodiments, the neural network 500 (e.g., a feedforward neural network) is trained to predict VCD and glucose concentration, using the historical glucose and lactate measured concentrations. Other offline and operation measurements may also be used as inputs for training the neural network 500.

In other embodiments, a second- or higher-order, linear or non-linear regression model is used as the predictive model 142 and/or 206. For a second-order model, the current cell culture attribute measurements and the measurements from an earlier time interval (e.g., the previous day) may be stored in vectors a and b, respectively. These two vectors, along with their different combinations with predicted values (e.g., as in the sequence 400 of FIG. 4), are the inputs to the regression model. Vectors a and b can be combined to the form input vector x(k) for the second-order model.

For a third-order model, the input structure is similar to the input of the second-order model. However, the third-order model input further includes measurements from a still earlier time interval (e.g., the day before the previous day), as vector c. Similarly, this increases the input dimension of the regression model. Vectors a, b, and c are combined to form input vector x(k) for the third-order model.

Combining the second- and third-order models enables predictions of metabolites starting from second day of the experiment (i.e., use of Day 0 and Day 1 data via the second-order model), with increased forecasting accuracy by using the third-order model starting from Day 3 of the experiment.

Assuming x(k) to be the input vector for the regression model and ŷ(k+1) as an output (prediction) of the regression model, the governing equation of the regression model may be described as:

ŷ(k+1)=αx(k)+e(k) (Equation 3)

In Equation 3, α is a vector of model coefficients and e(k) represents the identification error and measurement noise. The cost function

$$J = \frac{1}{2} {\sum }_{k = 1}^{N} {‖ y ( k ) - \hat{y} ( k ) ‖}^{2} + {‖ α ‖}_{2}^{2} \qquad ( Equation 4 )$$

Adding ∥α∥₂² (the second norm of α, squared) to the cost function in Equation 4 reduces the effect of unnecessary regressors on the output of the model, and leverages efforts for future model pruning (i.e., reducing the number of model inputs).

Training of the predictive model, whether a neural network or a regression model, can be challenging given the limited availability of granular, real-world historical data from cell culture processes. Metabolite concentrations may not have been measured and recorded each day, for example. In some embodiments, therefore, linear interpolation is used to provide more data points (i.e., “missing” values) for a larger training data set, although such interpolation tends to be inaccurate. In some embodiments, the predictive model (e.g., predictive model 114 and/or 206) continuously adapts by using measured and predicted values of a cell culture attribute as labels and inputs, respectively, in subsequent training of the predictive model (i.e., after the predictive model has been initially trained and put into use). In this manner, the predictive accuracy may continue to increase over time.

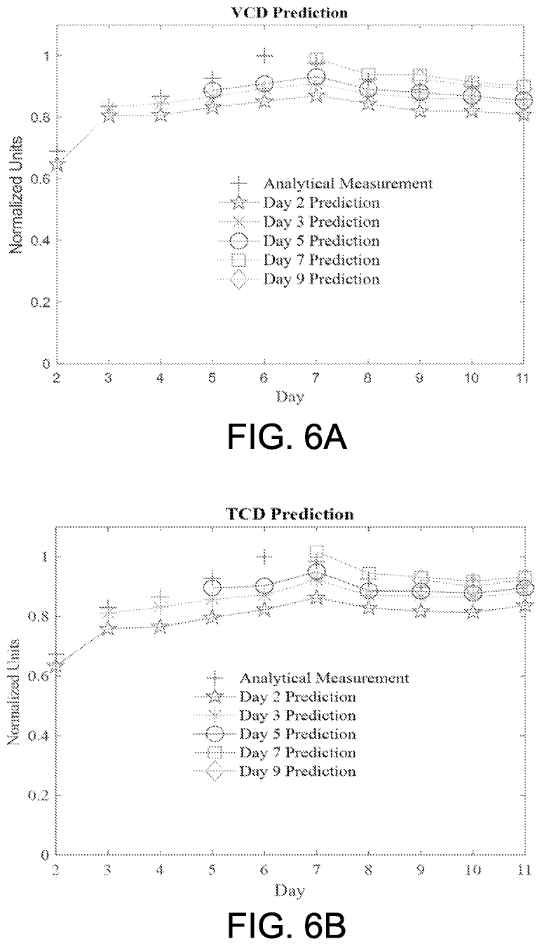

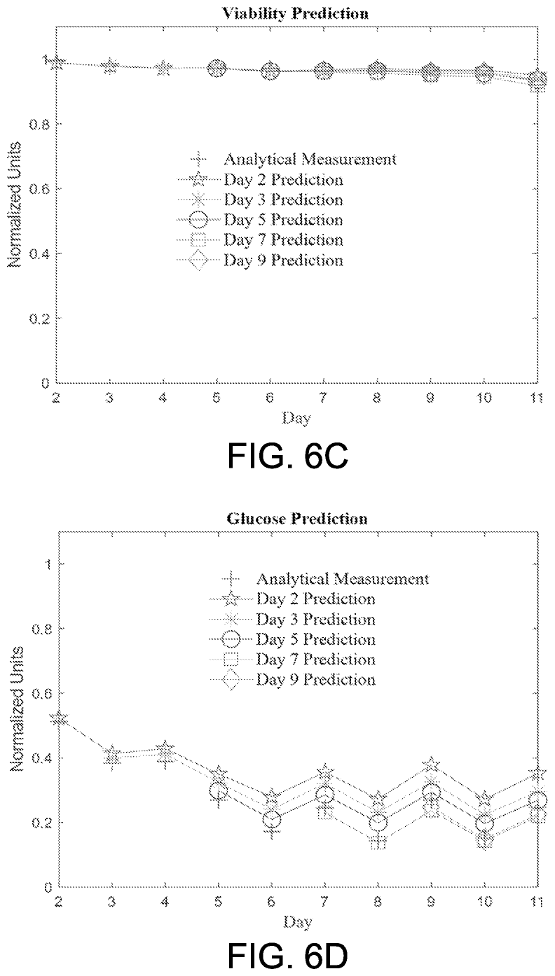

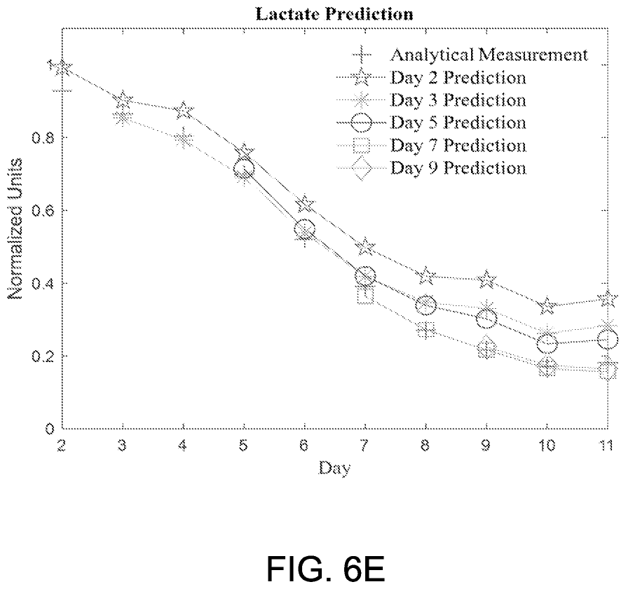

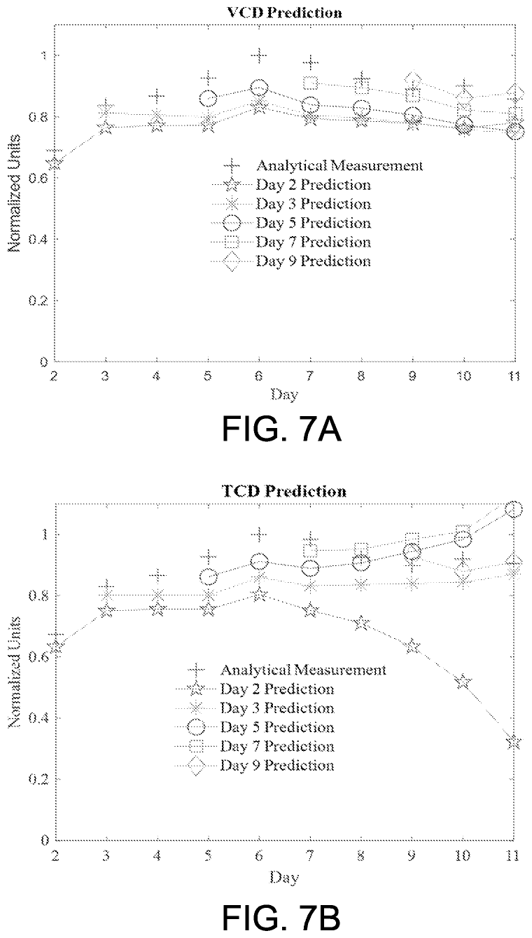

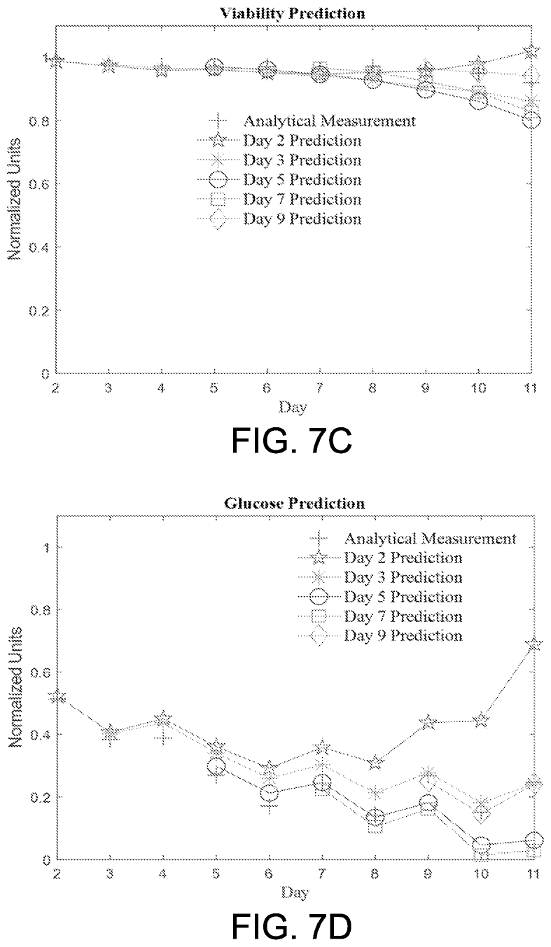

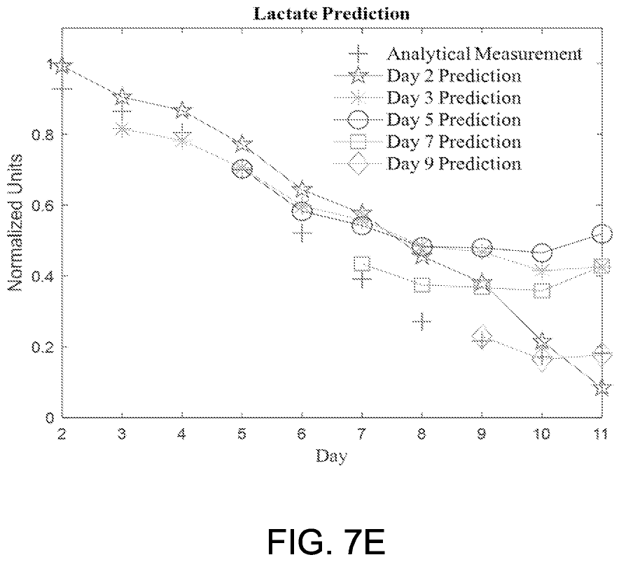

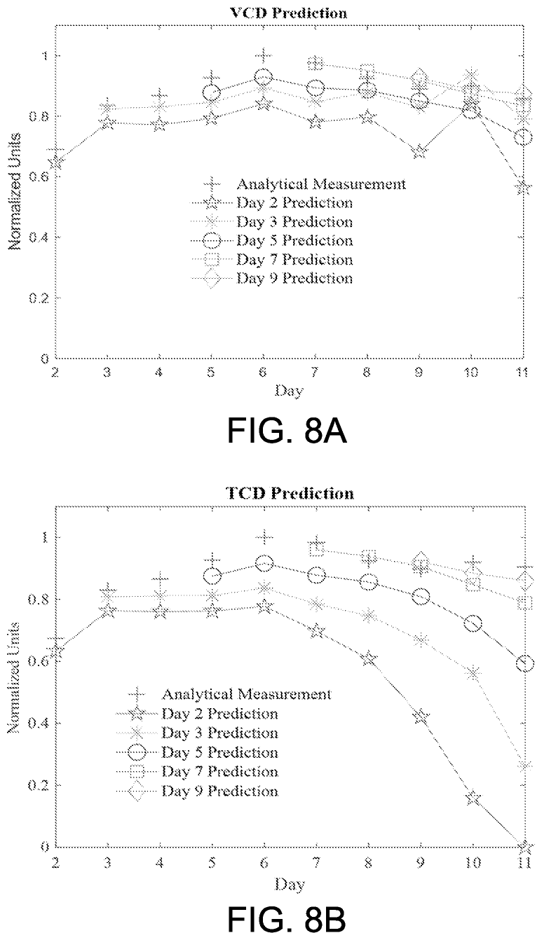

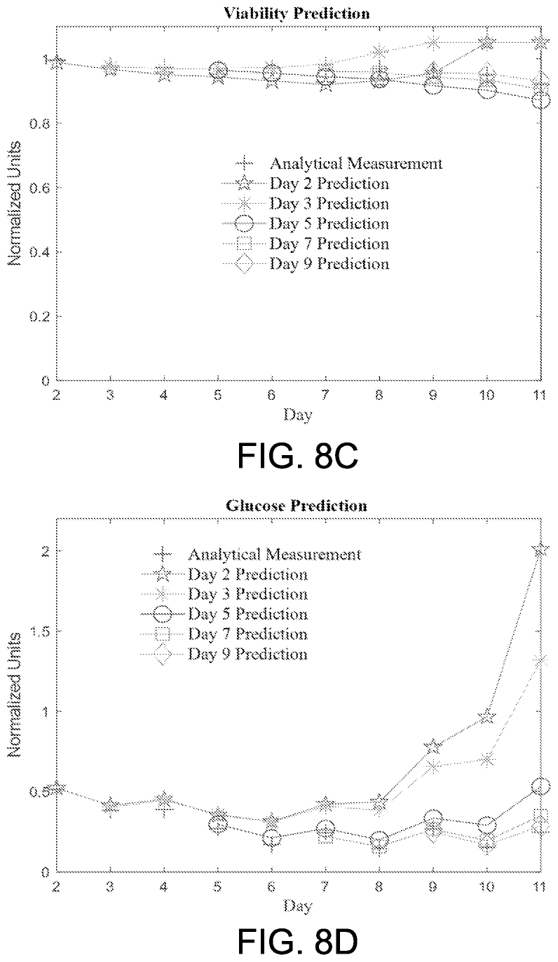

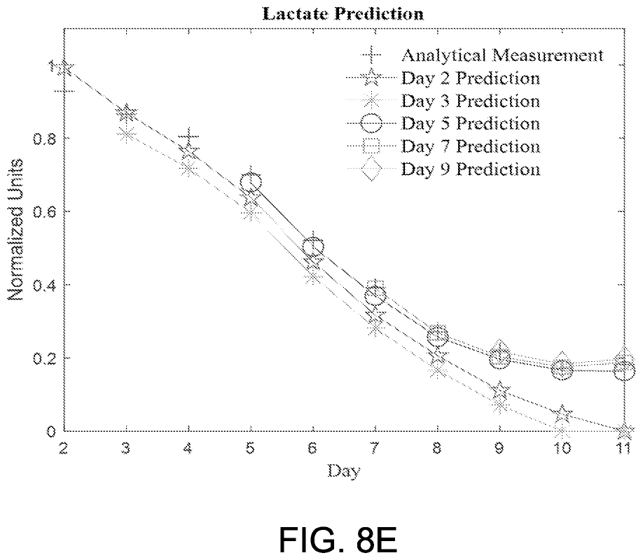

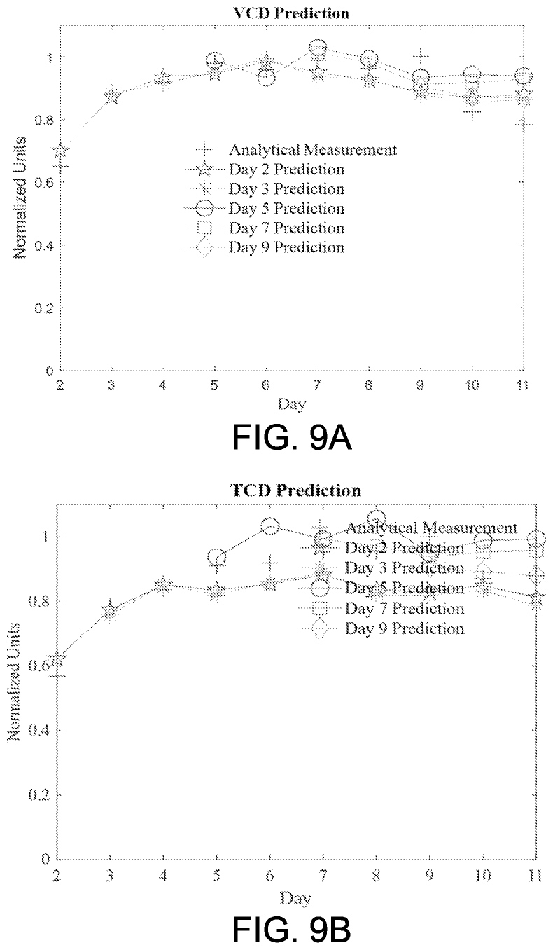

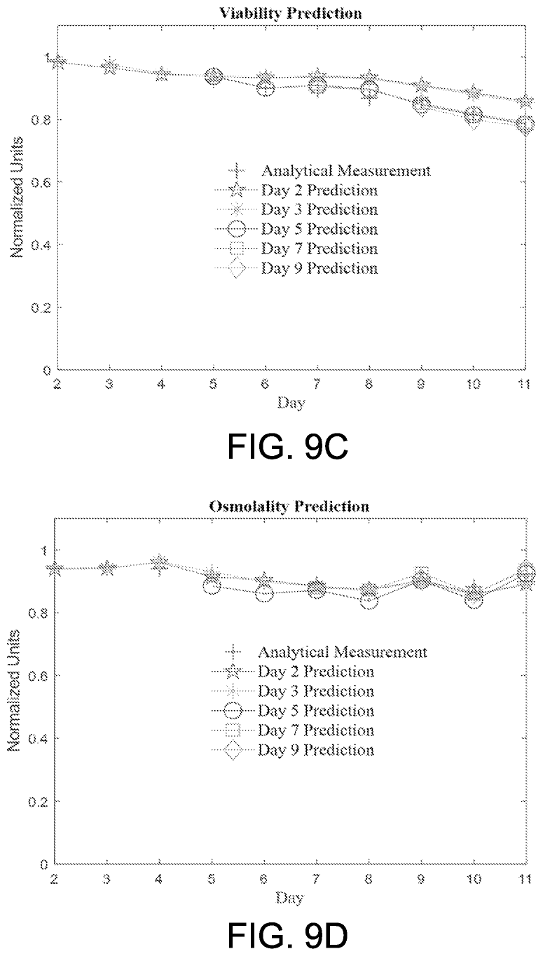

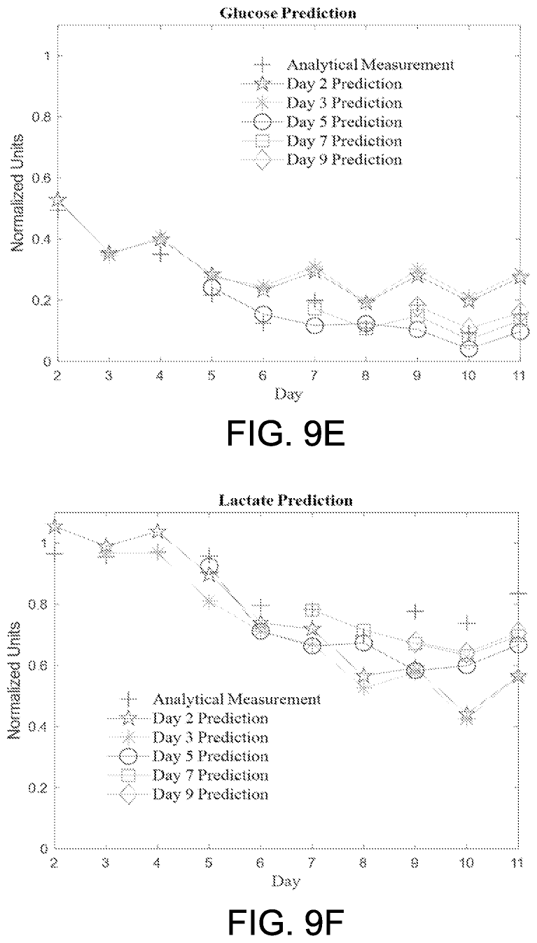

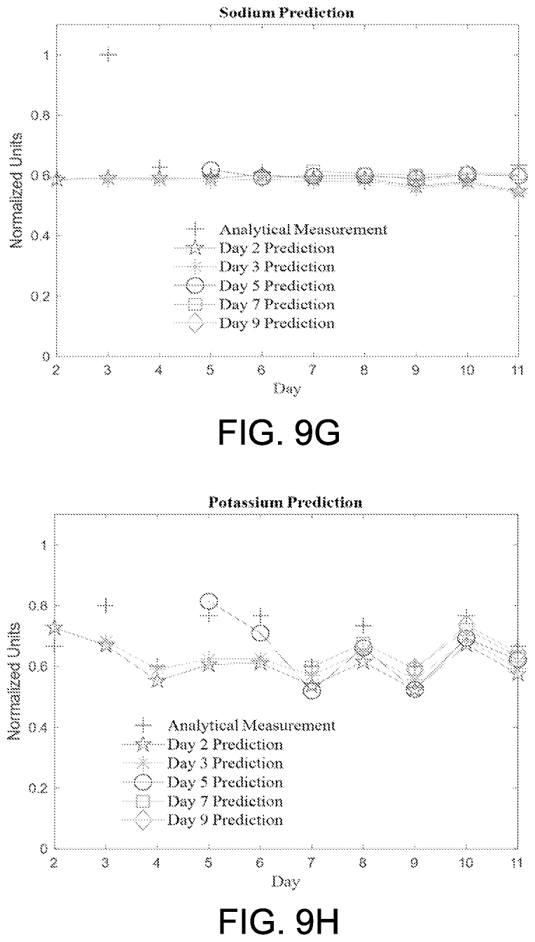

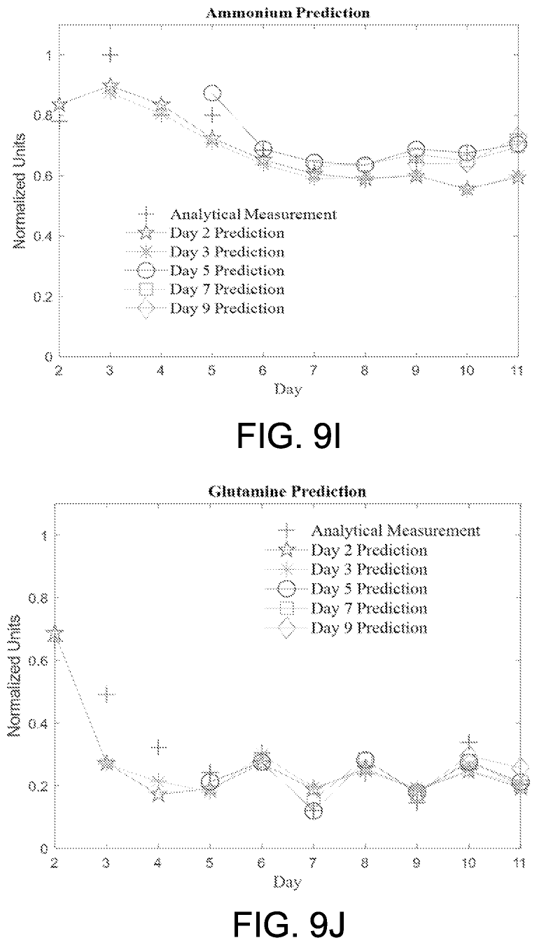

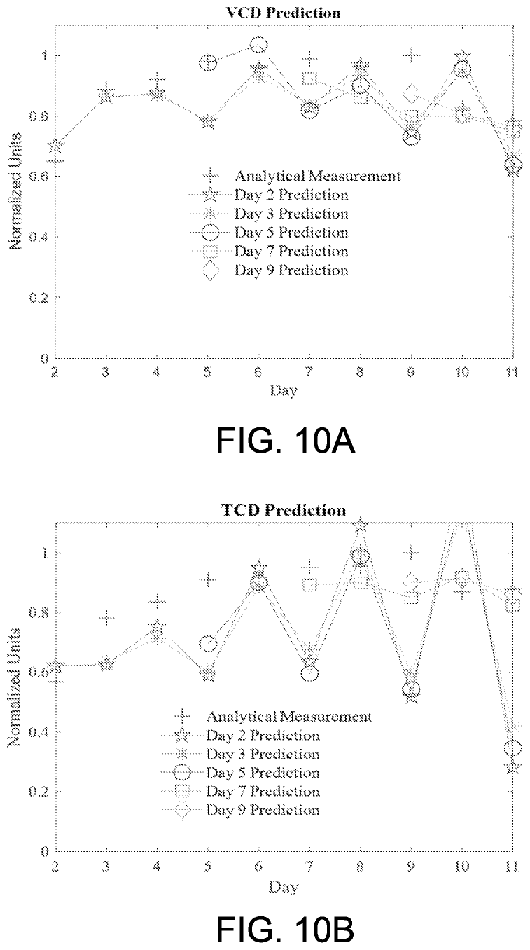

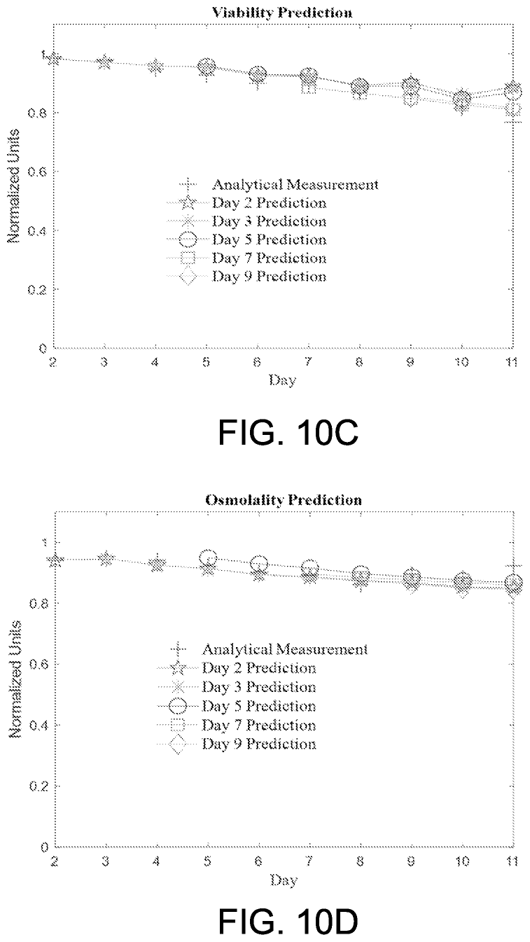

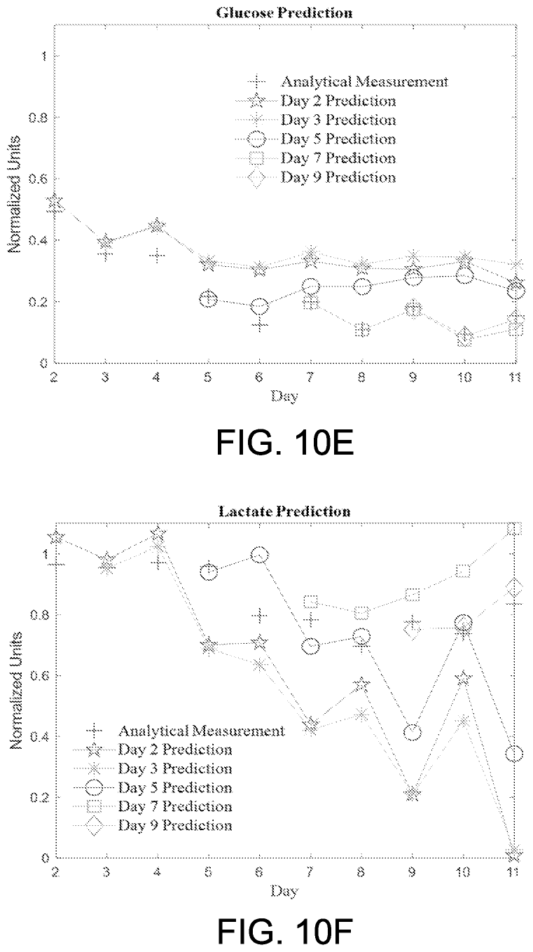

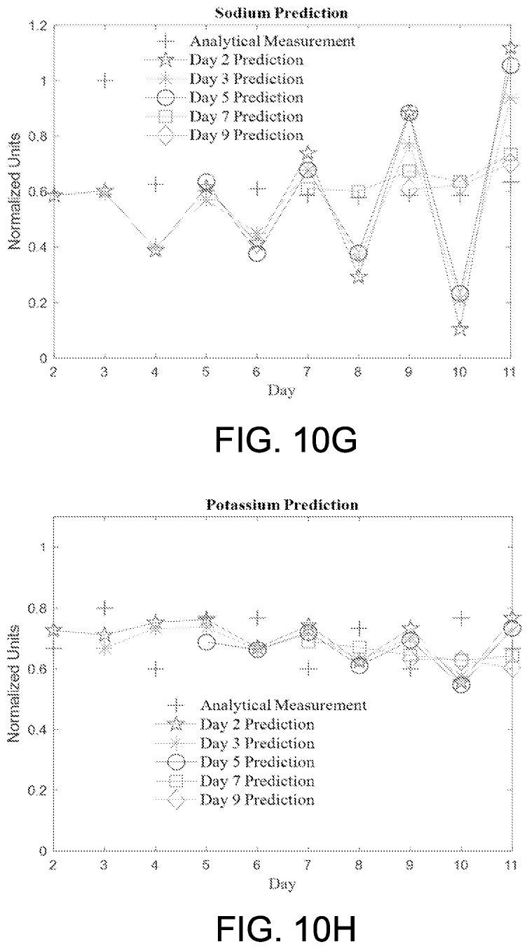

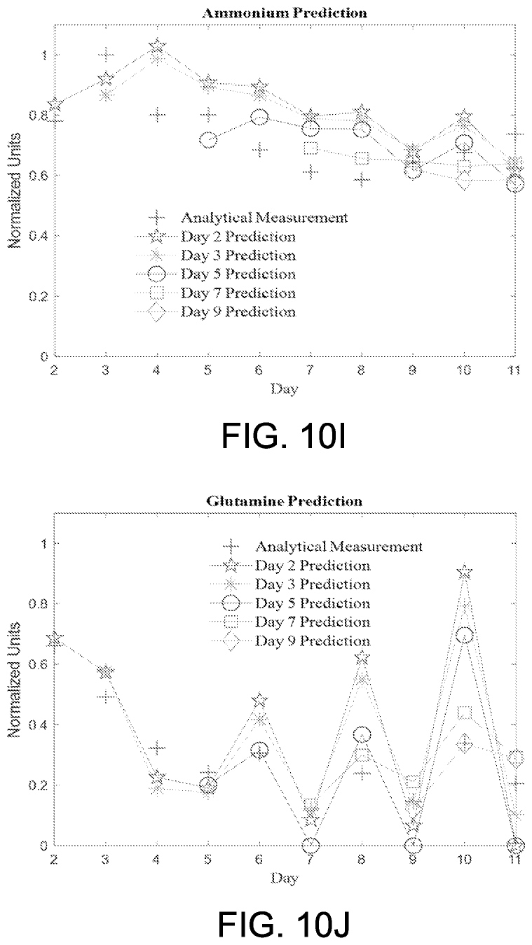

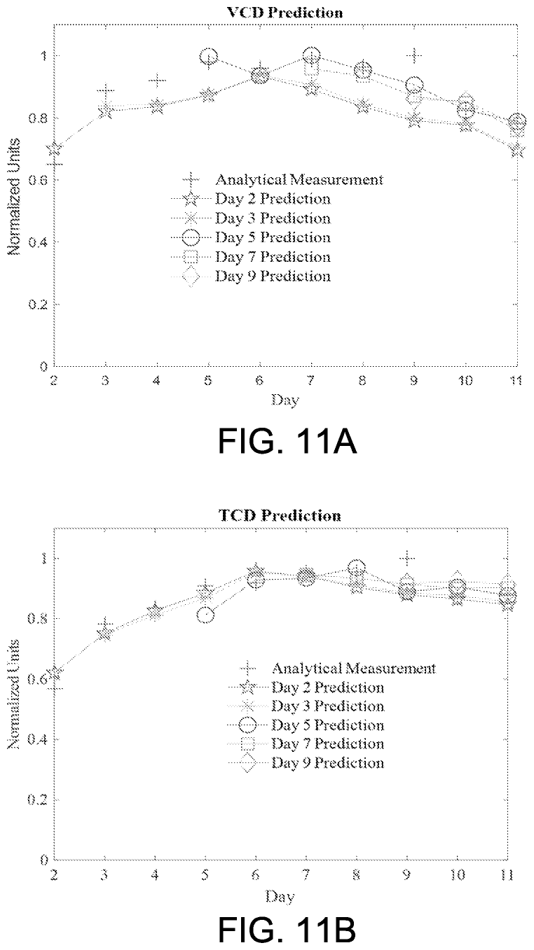

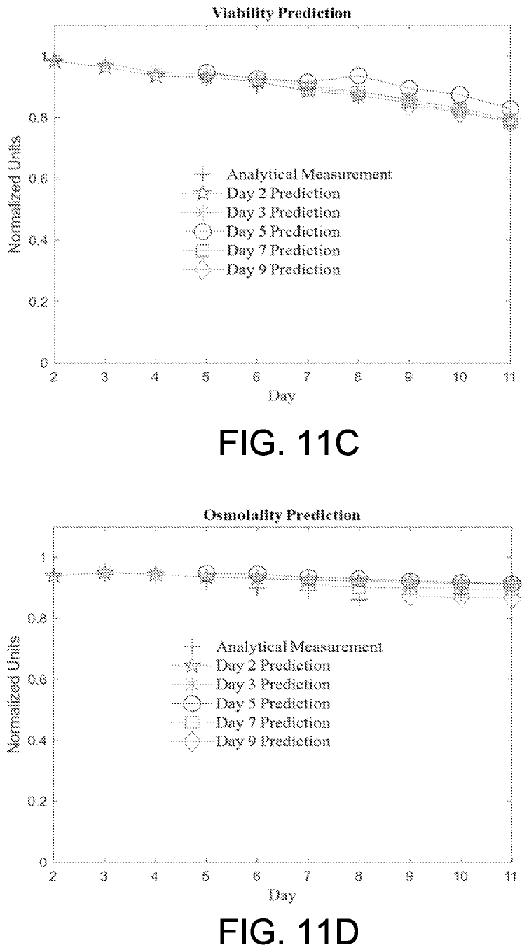

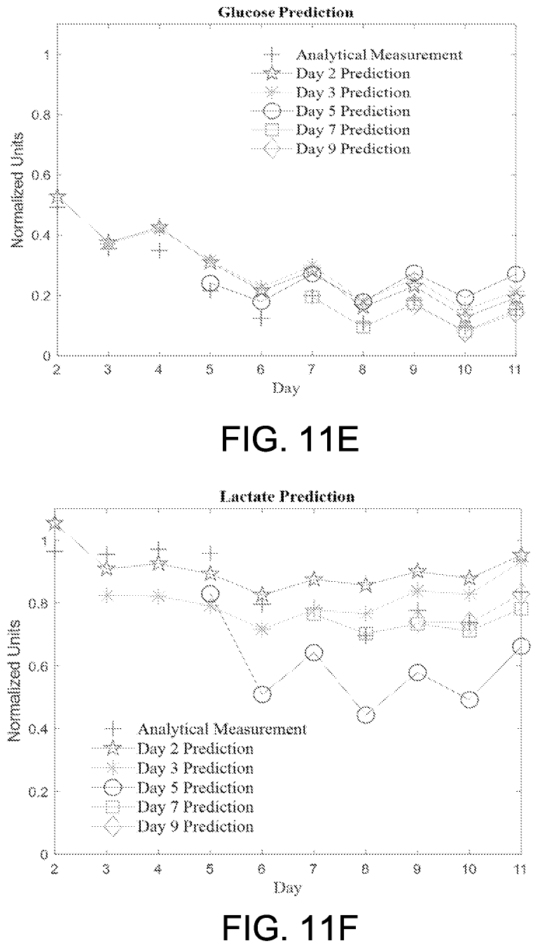

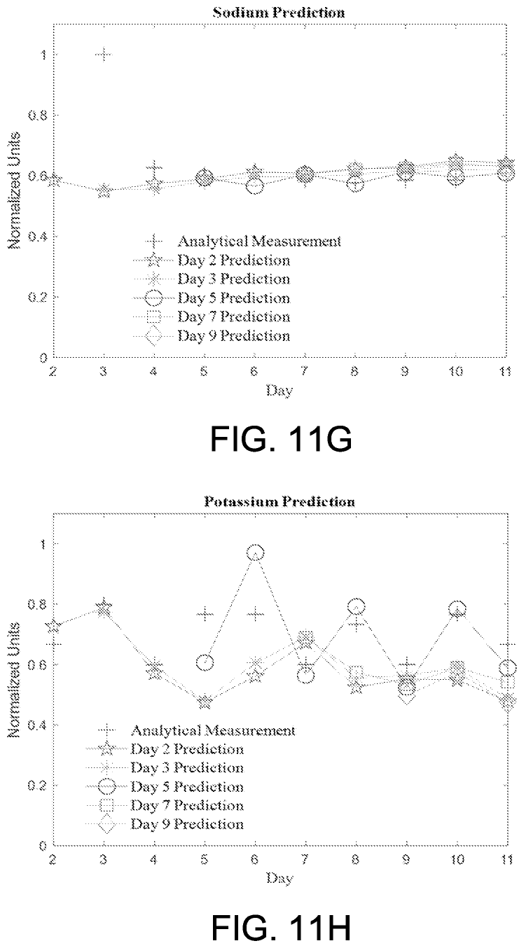

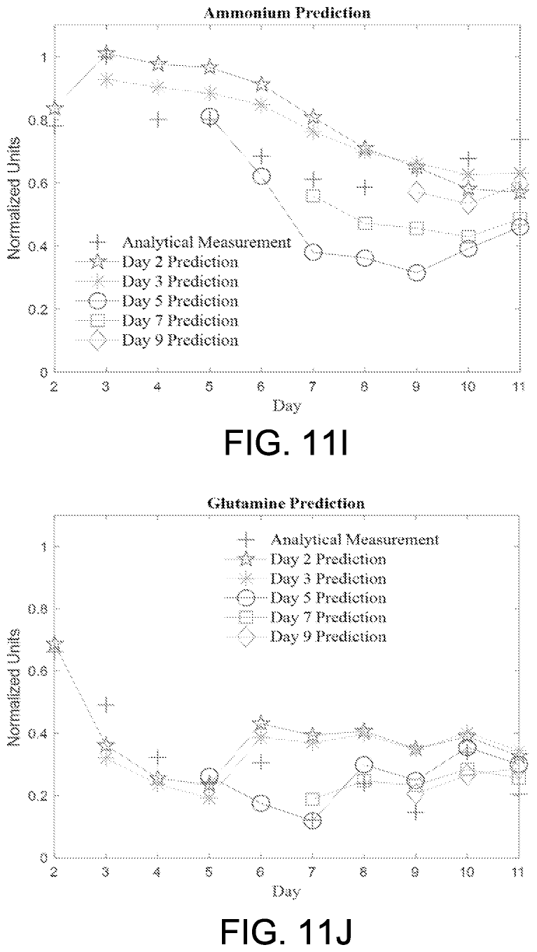

FIGS. 6-11 illustrate the performance of various embodiments of the intelligent control techniques discussed herein. Specifically, FIGS. 6A-6E are example plots comparing measured and predicted values of different cell culture attributes when using a neural network and a truncated set of metabolite measurements, FIGS. 7A-7E are example plots comparing measured and predicted values of different cell culture attributes when using a second-order regression model and a truncated set of metabolite measurements, and FIGS. 8A-8E are example plots comparing measured and predicted values of different cell culture attributes when using a third-order regression model and a truncated set of metabolite measurements. FIGS. 9A-9J are example plots comparing measured and predicted values of different cell culture attributes when using a neural network and a full set of metabolite measurements, FIGS. 10A-10J are example plots comparing measured and predicted values of different cell culture attributes when using a second-order regression model and a full set of metabolite measurements, and FIGS. 11A-11J are example plots comparing measured and predicted values of different cell culture attributes when using a third-order regression model and a full set of metabolite measurements.

In each plot of FIGS. 6-11, analytical measurements of real-world values for the indicated cell culture attribute (e.g., VCD in FIG. 6A, TCD in FIG. 6B, etc.) are depicted as plus (“+”) signs, and traces labeled as “Day z prediction” (e.g., Day 3, Day 5, etc.) correspond to the case where the analytical measurements up to Day z−1 have been used (e.g., by predictive model 114 or 206) to predict the value of the indicated cell culture attribute from Day z through the final day of the run (in these examples, Day 11). Each plot in FIGS. 6A-6E, and each plot in FIGS. 7A-7E, etc., corresponds to predictions made by a different model (e.g., such that a first neural network predicts VCD in FIG. 6A, a second neural network predicts TCD in FIG. 6B, etc.). For the various models of FIGS. 6-11, predictions of values corresponding to subsequent time intervals (e.g., k+2, etc.) are made by combining measured and predicted values in a manner similar to the sequence 400 depicted in FIG. 4.

As can be seen in FIGS. 7A-7E and 8A-8E (as well as FIGS. 10A-10J and 11A-11J), the second-order regression model allows predictions to be made starting at Day 2, while the third-order regression model allows predictions to be made starting at Day 3. FIGS. 6A-6E (as well as FIGS. 9A-9J) reflect a third-order neural network, and thus predictions are made starting at Day 3. For the second-order and third-order regression models, unconstrained multivariate minimization was used as the training algorithm. In all cases, a bioreactor volume of one liter was used to train and test the model, and the training data set size was increased using linear interpolation. Measured variables were concatenated into a vector to represent the model inputs.

For FIGS. 9-11, the basic model structures were unchanged relative to FIGS. 6-8, respectively. However, more parameters (cell culture attributes) were measured or otherwise obtained for modeling (specifically, feed volume, VCD, TCD, viability, lactate concentration, glucose concentration, sodium concentration, potassium concentration, ammonium concentration, and glutamine concentration). While most of these parameters are measured values, feed volume can be taken from the control set-point or can be measured. Using measurements of this larger set of metabolites increases the input and model dimension, which in turn requires more training data to train each model effectively. However, the resulting, trained models may more accurately predict metabolite levels and/or other cell culture attribute values, as seen in FIGS. 6-11.

As seen in FIGS. 7A-7E and 10A-10J, for the second-order regression model, prediction quality/accuracy generally improves over time. However, prediction accuracy for a second-order model is generally less than the prediction accuracy of a neural network (FIGS. 6A-6E, 9A-9J) or a third-order regression model (FIGS. 8A-8E, 11A-11J).

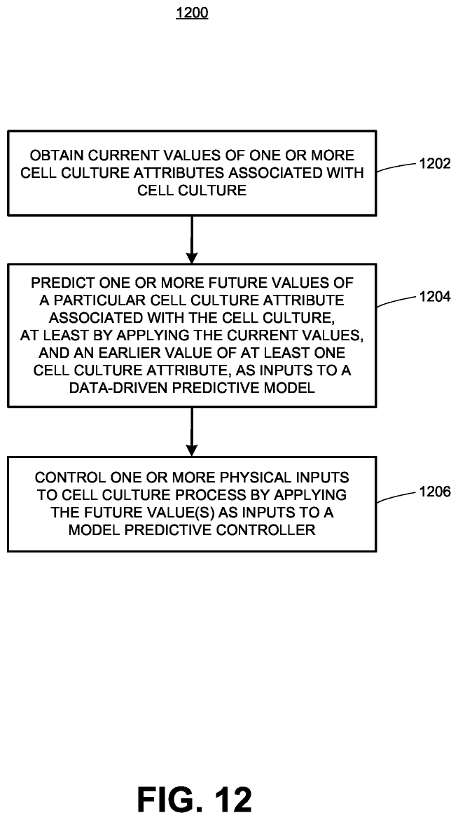

FIG. 12 is a flow diagram of an example method 1200 of controlling a cell culture process. The method 1200 may be implemented by a system such as the system 100 of FIG. 1 (e.g., by the processing hardware 120 executing instructions of the CCP control application 130, and/or by the model server 108). The method 1200 may be repeated (e.g., in real-time) for one or more time intervals (e.g., each of multiple days) during the cell culture process.

At block 1202, current values of one or more cell culture attributes associated with a cell culture (e.g., in a bioreactor such as bioreactor 102) are obtained from manual sampling or a simulation. Block 1202 may include receiving the current values from another device or system (e.g., from analytical instrument(s) 104), directly measuring some or all of the values (e.g., by analytical instrument(s) 104), and/or inferring or predicting some or all of the values (e.g., based on Raman spectroscopy measurements/scan vectors and using JITL), for example. The cell culture attributes for which values are obtained may include one or more metabolite levels (e.g., concentrations), VCD, TCD, viability, added feed volume, and/or one or more other attributes of the cell culture.

At block 1204, one or more future values of a particular cell culture attribute associated with the cell culture are predicted. Block 1204 includes applying the current values, and at least one earlier value of at least one cell culture attribute, as inputs to a data-driven predictive model (e.g., the predictive model 114 or 206). The earlier value(s) of the cell culture attribute(s) may be value(s) obtained from manual sampling, or a simulation, that occurred at an earlier time interval, for example. In some embodiments, for example, both the current value(s) of block 1202 and the earlier value(s) of block 1204 are/were obtained by using JITL to infer or predict those values based on Raman spectroscopy measurements/scan vectors. The predictive model may be a neural network (e.g., a feedforward neural network) or a regression model of at least second-order (e.g., third-order). The particular cell culture attribute for which the future value(s) are predicted may include a metabolite level (e.g., concentration), VCD, TCD, viability, or a different attribute of the cell culture. The number of future values predicted generally depends on the desired finite control horizon, which may be any suitable length (e.g., four days, or any suitable length between two and six days, etc.).

At block 1206, one or more physical inputs to the cell culture process are controlled. Block 1206 includes applying the future value(s) (predicted at block 1204) as inputs to an MPC. The MPC may output a value that is used (e.g., by CCP control application 130) to generate a control signal (e.g., a command specifying a set-point) to be sent to an input device (control element), such as one of input device(s) 112.

In some embodiments, the method 1200 includes one or more additional blocks not shown in FIG. 12. For example, blocks similar to blocks 1204 and 1206 (and possibly also block 1202) may be performed in parallel with respect to one or more other predictive models that predict future values of other cell culture attributes.

Additional considerations pertaining to this disclosure will now be addressed.

Some of the figures described herein illustrate example block diagrams having one or more functional components. It will be understood that such block diagrams are for illustrative purposes and the devices described and shown may have additional, fewer, or alternate components than those illustrated. Additionally, in various embodiments, the components (as well as the functionality provided by the respective components) may be associated with or otherwise integrated as part of any suitable components.

Embodiments of the disclosure relate to a non-transitory computer-readable storage medium having computer code thereon for performing various computer-implemented operations. The term “computer-readable storage medium” is used herein to include any medium that is capable of storing or encoding a sequence of instructions or computer codes for performing the operations, methodologies, and techniques described herein. The media and computer code may be those specially designed and constructed for the purposes of the embodiments of the disclosure, or they may be of the kind well known and available to those having skill in the computer software arts. Examples of computer-readable storage media include, but are not limited to: magnetic media such as hard disks, floppy disks, and magnetic tape; optical media such as CD-ROMs and holographic devices; magneto-optical media such as optical disks; and hardware devices that are specially configured to store and execute program code, such as ASICs, programmable logic devices (“PLDs”), and ROM and RAM devices.

Examples of computer code include machine code, such as produced by a compiler, and files containing higher-level code that are executed by a computer using an interpreter or a compiler. For example, an embodiment of the disclosure may be implemented using Java, C++, or other object-oriented programming language and development tools. Additional examples of computer code include encrypted code and compressed code. Moreover, an embodiment of the disclosure may be downloaded as a computer program product, which may be transferred from a remote computer (e.g., a server computer) to a requesting computer (e.g., a client computer or a different server computer) via a transmission channel. Another embodiment of the disclosure may be implemented in hardwired circuitry in place of, or in combination with, machine-executable software instructions.

As used herein, the singular terms “a,” “an,” and “the” may include plural referents, unless the context clearly dictates otherwise.

As used herein, the terms “approximately,” “substantially,” “substantial” and “about” are used to describe and account for small variations. When used in conjunction with an event or circumstance, the terms can refer to instances in which the event or circumstance occurs precisely as well as instances in which the event or circumstance occurs to a close approximation. For example, when used in conjunction with a numerical value, the terms can refer to a range of variation less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. For example, two numerical values can be deemed to be “substantially” the same if a difference between the values is less than or equal to ±10% of an average of the values, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified.

While the present disclosure has been described and illustrated with reference to specific embodiments thereof, these descriptions and illustrations do not limit the present disclosure. It should be understood by those skilled in the art that various changes may be made and equivalents may be substituted without departing from the true spirit and scope of the present disclosure as defined by the appended claims. The illustrations are not necessarily drawn to scale. There may be distinctions between the artistic renditions in the present disclosure and the actual apparatus due to manufacturing processes, tolerances and/or other reasons. There may be other embodiments of the present disclosure which are not specifically illustrated. The specification (other than the claims) and drawings are to be regarded as illustrative rather than restrictive. Modifications may be made to adapt a particular situation, material, composition of matter, technique, or process to the objective, spirit and scope of the present disclosure. All such modifications are intended to be within the scope of the claims appended hereto. While the techniques disclosed herein have been described with reference to particular operations performed in a particular order, it will be understood that these operations may be combined, sub-divided, or re-ordered to form an equivalent technique without departing from the teachings of the present disclosure. Accordingly, unless specifically indicated herein, the order and grouping of the operations are not limitations of the present disclosure.

1. A method of controlling a cell culture process, the method comprising, for one or more time intervals during the cell culture process:
obtaining current values, from manual sampling or a simulation, of one or more cell culture attributes associated with a cell culture;
predicting, by processing hardware, one or more future values of a particular cell culture attribute associated with the cell culture, at least by applying (i) the current values of the one or more cell culture attributes, and (ii) an earlier value, obtained from manual sampling or a simulation at an earlier time interval, of at least one of the one or more cell culture attributes, as inputs to a data-driven predictive model; and
controlling, by the processing hardware applying the one or more future values as inputs to a model predictive controller, one or more physical inputs to the cell culture process.

2. The method of claim 1, wherein the data-driven predictive model is a regression model.

3. The method of claim 2, wherein:
obtaining the current values includes obtaining the current values during a current time interval; and
predicting the one or more future values of the particular cell culture attribute includes, for a first cell culture attribute of the one or more cell culture attributes, applying (i) the current value of the first cell culture attribute, (ii) a value of the first cell culture attribute that was obtained during a first previous time interval that occurred prior to the current time interval, and (iii) a value of the first cell culture attribute that was obtained during a second previous time interval that occurred prior to the first previous time interval, as inputs to the data-driven predictive model.

4. The method of claim 3, further comprising, for one or more additional time intervals that occur prior to the one or more time intervals:
obtaining additional current values of the one or more cell culture attributes during a current time interval;
predicting, by the processing hardware, an additional one or more future values of the particular cell culture attribute, at least by applying (i) the additional current values of the one or more cell culture attributes, (ii) an additional value of the first cell culture attribute that was obtained as an input to a different regression model; and
controlling, by the processing hardware applying the additional one or more future values as inputs to the model predictive controller.

5. The method of claim 1, wherein the data-driven predictive model is a neural network.

6. The method of claim 5, wherein the neural network is a feedforward neural network.

7. The method of claim 1, wherein obtaining the current values of the one or more cell culture attributes associated with the cell culture includes:
obtaining one or more Raman spectroscopy measurements of the cell culture; and
determining the current value of at least one of the one or more cell culture attributes based on the one or more Raman spectroscopy measurements.

8. The method of claim 1, wherein:
the one or more cell culture attributes include one or more of (i) one or more metabolite levels, (ii) viable cell density, (iii) total cell density, (iv) viability, or (v) added feed volume; and
the particular cell culture attribute is one of (i) a metabolite level, (ii) viable cell density, (iii) total cell density, or (iv) viability.

9. The method of claim 1, wherein predicting the one or more future values of the particular cell culture attribute includes predicting a future value for each of at least two different days.

10. The method of claim 1, wherein controlling the one or more physical inputs to the cell culture process includes controlling an amount of glucose introduced into the cell culture.

11. One or more non-transitory, computer-readable media storing instructions that, when executed by processing hardware of a computing system and for one or more time intervals during a cell culture process, cause the computing system to:
obtain current values, from manual sampling or a simulation, of one or more cell culture attributes associated with a cell culture;
predict one or more future values of a particular cell culture attribute associated with the cell culture, at least by applying (i) the current values of the one or more cell culture attributes, and (ii) an earlier value, obtained from manual sampling or a simulation at an earlier time interval, of at least one of the one or more cell culture attributes, as inputs to a data-driven predictive model; and
control, by applying the one or more future values as inputs to a model predictive controller, one or more physical inputs to the cell culture process.

12. The one or more non-transitory, computer-readable media of claim 11, wherein the data-driven predictive model is a regression model.

13. The one or more non-transitory, computer-readable media of claim 12, wherein:
obtaining the current values includes obtaining the current values during a current time interval; and
predicting the one or more future values of the particular cell culture attribute includes, for a first cell culture attribute of the one or more cell culture attributes, applying (i) the current value of the first cell culture attribute, (ii) a value of the first cell culture attribute that was obtained during a first previous time interval that occurred prior to the current time interval, and (iii) a value of the first cell culture attribute that was obtained during a second previous time interval that occurred prior to the first previous time interval, as inputs to the data-driven predictive model.

14. The one or more non-transitory, computer-readable media of claim 13, wherein the data-driven predictive model is a neural network.

15. The one or more non-transitory, computer-readable media of claim 11, wherein:
the one or more cell culture attributes include one or more of (i) one or more metabolite levels, (ii) viable cell density, (iii) total cell density, (iv) viability, or (v) added feed volume; and
the particular cell culture attribute is one of (i) a metabolite level, (ii) viable cell density, (iii) total cell density, or (iv) viability.

16. The one or more non-transitory, computer-readable media of claim 11, wherein controlling the one or more physical inputs to the cell culture process includes controlling an amount of glucose introduced into the cell culture.

17. A system comprising:
a bioreactor configured to hold a cell culture during a cell culture process;
one or more electronically-controllable input devices configured to provide physical inputs to the cell culture process;
one or more analytical instruments configured to measure one or more cell culture attributes associated with the cell culture; and
a computing system configured to, for one or more time intervals during the cell culture process,
predict one or more future values of a particular cell culture attribute associated with the cell culture, at least by applying (i) current values, from manual sampling or a simulation, of the one or more cell culture attributes, and (ii) an earlier value, obtained from manual sampling or a simulation at an earlier time interval, of at least one of the one or more cell culture attributes, as inputs to a data-driven predictive model, and
control one or more physical inputs to the cell culture process, at least by applying the one or more future values as inputs to a model predictive controller to generate one or more control set-points for the one or more electronically-controllable input devices.

18. The system of claim 17, wherein the data-driven predictive model is a regression model.

19. The system of claim 18, wherein:
obtaining the current values includes obtaining the current values during a current time interval; and
predicting the one or more future values of the particular cell culture attribute includes, for a first cell culture attribute of the one or more cell culture attributes, applying (i) the current value of the first cell culture attribute, (ii) a value of the first cell culture attribute that was obtained during a first previous time interval that occurred prior to the current time interval, and (iii) a value of the first cell culture attribute that was obtained during a second previous time interval that occurred prior to the first previous time interval, as inputs to the data-driven predictive model.

20. The system of claim 19, wherein the data-driven predictive model is a neural network.

21. The system of claim 17, wherein:
the one or more cell culture attributes include one or more of (i) one or more metabolite levels, (ii) viable cell density, (iii) total cell density, (iv) viability, or (v) added feed volume; and
the particular cell culture attribute is one of (i) a metabolite level, (ii) viable cell density, (iii) total cell density, or (iv) viability.

22. The system of claim 17, wherein:
the one or more electronically-controllable input devices include a glucose pump; and
controlling the one or more physical inputs to the cell culture process includes controlling an amount of glucose introduced into the cell culture via the glucose pump.
